(19) United States
(12) Patent Application Publication
(10) Pub. No.: US 2023/0240606 A1
(43) Pub. Date: Aug. 3, 2023

(54) Monitoring Psychomotor Performance Based on Eyelid Tracking Information

(71) Applicant: Zinn Labs, Inc., Palo Alto, CA (US)

(72) Inventors: Kevin Boyle, San Francisco, CA (US); Robert Konrad, San Francisco, CA (US); Nitish Padmanaban, Menlo Park, CA (US)

(21) Appl. No.: 18/156,843

(22) Filed: Jan. 19, 2023

Related U.S. Application Data
(60) Provisional application No. 63/304,764, filed on Jan. 31, 2022.
(60) Provisional application No. 63/345,398, filed on May 24, 2022.

(51) Int. Cl. A61B 5/00; A61B 5/16

(52) U.S. Cl. CPC A61B 5/6803; A61B 5/163; A61B 5/4806; A61B 5/0082

(57) ABSTRACT

Embodiments are related to a system with a headset capable of monitoring psychomotor performance of a user of the headset based on eyelid tracking information. The headset includes a sensor assembly coupled to a frame of the headset, and a transceiver coupled to the sensor assembly. The sensor assembly is configured to track an eyelid of an eye of the user and capture eyelid tracking information. The transceiver is configured to obtain the eyelid tracking information from the sensor assembly and communicate the eyelid tracking information to a secondary device coupled to the headset for processing the eyelid tracking information and determination of sleep information for the user based in part on the processed eyelid tracking information.

### CROSS REFERENCE TO RELATED APPLICATIONS

This application claims a priority and benefit to U.S. Provisional Patent Application Ser. No. 63/304,764, filed Jan. 31, 2022, and U.S. Provisional Patent Application Ser. No. 63/345,398, filed May 24, 2022, each of which is hereby incorporated by reference in its entirety.

### FIELD OF THE INVENTION

This disclosure relates generally to a system with a headset, and more specifically to a system for monitoring psychomotor performance for a user of the headset based on eyelid tracking information.

### BACKGROUND

There is currently no standardized hardware for eye-based health and wellness diagnostics. For example, a virtual reality gear with generic eye-tracking capability may be used for brain health diagnostics. An eye-tracking tablet can be used for, e.g., dynamic vision training. A smartphone camera can be utilized for, e.g., measuring efficacy of pain relief medication. A computer camera can be used for, e.g., cognitive health diagnostics. A generic high-resolution camera can be used for, e.g., operational risk management and/or epilepsy diagnostics. Thus, there is a need for a health/wellness monitoring based on a wearable smart electronic eyeglasses with a small form factor that can provide eye-based health and wellness diagnostics.

### SUMMARY

Embodiments of the present disclosure relate to a system with a headset capable of monitoring psychomotor performance of a user of the headset based on eyelid tracking information. The headset includes a sensor assembly coupled to a frame of the headset, and a transceiver coupled to the sensor assembly. The sensor assembly is configured to track an eyelid of an eye of the user (i.e., occlusion and disocclusion of a pupil/iris of the user's eye) and capture eyelid tracking information. The transceiver is configured to obtain the eyelid tracking information from the sensor assembly and communicate the eyelid tracking information to a secondary device coupled to the headset for processing the eyelid tracking information and determination of sleep information for the user based in part on the processed eyelid tracking information.

Some embodiments of the present disclosure relate to a method for utilizing a headset as part of a system for monitoring psychomotor performance of a user of the headset based on eyelid tracking information. The method comprises: tracking an eyelid of an eye of the user by a sensor assembly coupled to a frame of the headset; capturing eyelid tracking information at the sensor assembly; and communicating the eyelid tracking information from the headset to a secondary device coupled to the headset for processing the eyelid tracking information and determination of sleep information for the user based in part on the processed eyelid tracking information.

Some embodiments of the present disclosure further relate to a method for utilizing a device coupled to a headset for monitoring psychomotor performance of a user of the headset based on eyelid tracking information. The method comprises: receiving, at the device from the headset, eyelid tracking information captured at the headset associated with an eyelid of an eye of a user of the headset; processing the received eyelid tracking information to determine sleep information for the user; and presenting the determined sleep information to one or more users of the device.

### BRIEF DESCRIPTION OF THE DRAWINGS

FIG. 1 is a perspective view of a headset, in accordance with one or more embodiments.

FIG. 2 illustrates an example top view of a frame of a headset, in accordance with one or more embodiments.

FIG. 3A illustrates an example headset with sensor assemblies clipped onto temples of a frame of the headset, in accordance with one or more embodiments.

FIG. 3B illustrates an example headset with a sensor assembly embedded into a frame of a headset, in accordance with one or more embodiments.

FIG. 3C illustrates an example headset with an interchangeable frame, in accordance with one or more embodiments.

FIG. 4A illustrates an example graph illustrating correlation between a blink duration and psychomotor performance for a first user, in accordance with one or more embodiments.

FIG. 4B illustrates an example graph illustrating correlation between a blink duration and psychomotor performance for a second user, in accordance with one or more embodiments.

FIG. 5A illustrates an example of eyelid tracking over time, in accordance with one or more embodiments.

FIG. 5B illustrates an example of eyelid metric, in accordance with one or more embodiments.

FIG. 6 illustrates an example graph of a sleep sensitivity as a function of a needed sleep duration, in accordance with one or more embodiments.

FIG. 7A illustrates an example graph illustrating psychomotor performance correlated with a sleep duration for a first user, in accordance with one or more embodiments.

FIG. 7B illustrates an example graph illustrating psychomotor performance correlated with a sleep duration for a second user, in accordance with one or more embodiments.

FIG. 8 illustrates an example healthcare platform with a headset, in accordance with one or more embodiments.

FIG. 9 is a block diagram of a healthcare platform that includes a headset, in accordance with one or more embodiments.

FIG. 10 is a flow chart illustrating a process performed at a headset for capturing eyelid tracking information used for evaluating psychomotor performance of a user of the headset, in accordance with one or more embodiments.

FIG. 11 is a flow chart illustrating a process performed at a secondary device for determining sleep information for a user of a headset coupled to the secondary device based on eyelid tracking information captured at the headset, in accordance with one or more embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

Headsets (e.g., smart electronic eyeglasses) can have various initial applications including but not limiting to, e.g., artificial reality applications, allowing a natural refocusing experience for presbyopes, playing audio, and capturing world-facing video to record events. A headset can include one or more sensors that continuously and/or intermittently record user's data. Electronics components of the headset (e.g., one or more controllers coupled to one or more sensors) can be leveraged to provide information about the user that has previously been untapped by the eyewear market. By utilizing one or more sensors in the headset, user's data can be gathered continuously and/or intermittently that can be later used for health and wellness diagnostic purposes. Thus, the headset can serve as part of a health monitoring system.

Embodiments presented herein relate to small, low-power, lightweight smart electronic eyeglasses (i.e., headset) in a traditional eyewear form factor with “all-day” wireless sensing and a wireless connection (e.g., Bluetooth or WiFi) to a secondary device (e.g., smartphone, smartwatch, tablet, desktop, etc.). The headset with a corresponding sensing assembly can measure eye metrics that relate to a user's cognitive or psychomotor performance (i.e., reaction time) and relates changes in the user's performance to sleep habits (e.g., individual sleep needs and sensitivity to lost sleep). The headset with sensor assembly presented herein is configured for wearable cognitive health/wellness tracking (e.g., tracking of sleep habits and fatigue). The secondary device may show, analyze, and explain data in an app and suggest to the user ways to improve his/her own sleep habits.

A health monitoring system presented herein includes at least the headset in communication with the secondary device. The sensor assembly of the headset monitors (e.g., tracks) where an eyelid of a user's eye is positioned (e.g., percent closed) over time. The sensor assembly may be implemented as one or more light emitting diode (LEDs) paired with a detector. The detector may be implemented as, e.g., a camera, one or more photodiodes, one or more event sensors, etc. The sensor assembly may be coupled to (or integrated into) a temple of the headset. Eyelid information tracked and captured by the sensor assembly may be provided to the secondary device for processing. Alternatively, the captured eyelid tracking information may be at least partially processed at the headset. Eyelid tracking information is information related to tracked (e.g., monitored) positions of the user's eyelid over time. Eyelid tracking information may include, e.g., information about an amount of occlusion over time of a pupil for the user's eye, information about a position of the user's eyelid overtime relative to a reference point (e.g., on the headset), some other information related to the user's eyelid, or some combination thereof. The secondary device may utilize the processed eyelid tracking information in combination with information from a sleep tracker of the user to estimate how sleep deprivation is affecting a reaction time of the user. The secondary device may present the analyzed information to the user (and to some other user(s) in communication with the secondary device). Alternatively, the headset may present the analyzed information to the user.

In some embodiments, the analyzed information can be further communicated from the secondary device to a server platform. The server platform can efficiently perform a large number of computations to, e.g., extract interesting statistics and/or features from the user's data captured at the headset and expose the extracted statistics and/or the features to third parties through, e.g., an Application Processing Interface (API) of the server platform. In one or more embodiments, the third parties can access user's data streams communicated from the secondary device to the server platform and build their own health related applications on top of the server platform's API to run their own diagnostics.

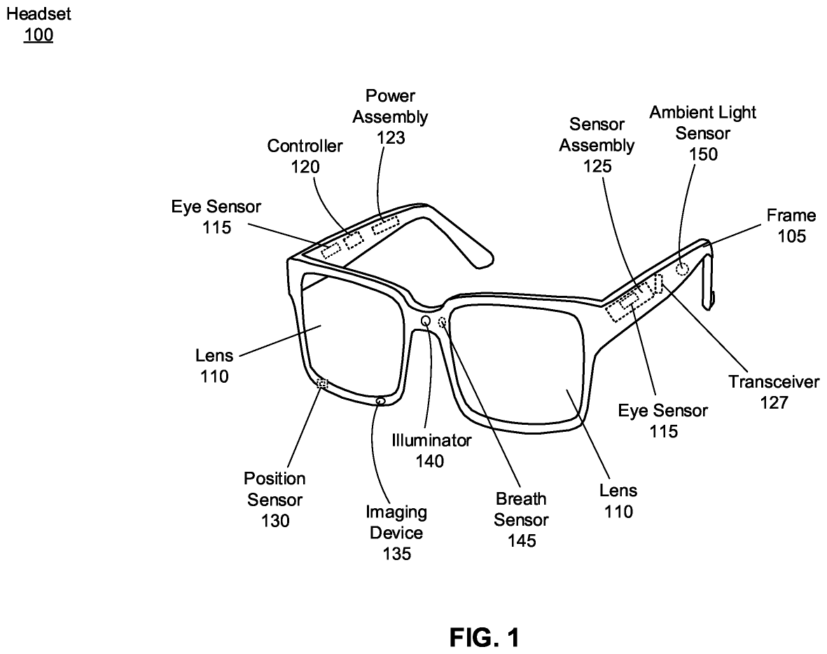

FIG. 1 is a perspective view of a headset 100, in accordance with one or more embodiments. In general, the headset 100 may be worn on the face of a user such that content (e.g., media content) is presented via one or more lenses 110 of the headset 100. However, the headset 100 may also be used such that media content is presented to a user in a different manner. Examples of media content presented by the headset 100 include one or more images, video, audio, or some combination thereof. The headset 100 may include, among other components, a frame 105, a pair of lenses 110, a plurality of various sensors, a depth camera assembly (DCA), a controller 120, a power assembly 123, and a transceiver 127. While FIG. 1 illustrates the components of the headset 100 in example locations on the headset 100, the components may be located elsewhere on the headset 100, on a peripheral device paired with the headset 100, or some combination thereof. Similarly, there may be more or fewer components on the headset 100 than what is shown in FIG. 1.

The headset 100 may correct or enhance the vision of a user, protect the eye of a user, or provide images to a user. The headset 100 may produce artificial reality content for the user. The headset 100 may be smart electronic eyeglasses. The headset 100 may be eyeglasses which correct for defects in a user's eyesight. The headset 100 may be sunglasses which protect a user's eye from the sun. The headset 100 may be safety glasses which protect a user's eye from impact. In some embodiments, one or more of a night vision device or infrared goggles to enhance a user's vision at night, a mask or full-face respirator that filters a user's air, a welding shield or helmet to protect a user's eyes from intense light and the user's face from sparks, a diving goggles that separate a user's eyes from surrounding water, etc., may include the functionality of the headset 100.

The frame 105 holds other components of the headset 100. The frame 105 includes a front part that holds the one or more lenses 110 and end pieces to attach to a head of the user. The front part of the frame 105 bridges the top of a nose of the user. The end pieces (e.g., temples) are portions of the frame 105 to which the temples of a user are attached. The length of the end piece may be adjustable (e.g., adjustable temple length) to fit different users. The end piece may also include a portion that curls behind the ear of the user (e.g., temple tip, earpiece).

The one or more lenses 110 provide light to a user wearing the headset 100. As illustrated, the headset 100 includes a lens 110 for each eye of the user. In some embodiments, each lens 110 is part of a display block (not shown in FIG. 1) that generates image light that is provided to an eye box of the headset 100. The eye box is a location in space that an eye of the user occupies while the user wears the headset 100. In this context, the headset 100 generates Virtual Reality (VR) content. In some embodiments, one or both of the lenses 110 are at least partially transparent, such that light from a local area surrounding the headset 100 may be combined with light from one or more display blocks to produce Augmented Reality (AR) and/or Mixed Reality (MR) content.

In some embodiments, the headset 100 does not generate image light, and each lens 110 transmits light from the local area to the eye box. For example, one or both of the lenses 110 may be a lens without correction (non-prescription) or a prescription lens (e.g., single vision, bifocal and trifocal, or progressive) to help correct for defects in a user's eyesight. In some embodiments, each lens 110 may be polarized and/or tinted to protect the user's eyes from the sun. In some embodiments, each lens 110 may have a light blocking feature being activated, e.g., each lens 110 may be implemented as an electrochromic lens. In some embodiments, the lens 110 may include an additional optics block (not shown in FIG. 1). The optics block may include one or more optical elements (e.g., lens, Fresnel lens, etc.) that direct light to the eye box. The optics block may, e.g., correct for aberrations in some or all of visual content presented to the user, magnify some or all of the visual content, or some combination thereof.

In some embodiments, the lens 110 operates as a varifocal optical element that change its focal distance based on a user's eye gaze, e.g., as a focus-tunable lens. The lens 110 may be implemented as a liquid lens, liquid crystal lens, or some other type of lens that is able to vary its optical power. The lens 110 may be directly coupled to the controller 120, and the controller 120 may provide appropriate varifocal instructions (e.g., pulses with various voltage levels) to at least one portion of the lens 110 in order to change at least one optical power associated with the at least one portion of the lens 110.

The DCA determines depth information for a portion of a local area surrounding the headset 100. The DCA includes one or more imaging devices 135 and a DCA controller (not shown in FIG. 1) and may also include one or more illuminators 140. In some embodiments, the illuminator 140 illuminates a portion of the local area with light. The light may be, e.g., structured light (e.g., dot pattern, bars, etc.) in the infrared (IR), IR flash for time-of-flight, etc. In some embodiments, the one or more imaging devices 135 capture images of the portion of the local area that include the light from the illuminator 140. As illustrated, FIG. 1 shows a single illuminator 140 and a single imaging device 135. In alternate embodiments, there are at least two imaging devices 135 integrated into the frame 105. The DCA controller computes depth information for the portion of the local area using the captured images and one or more depth determination techniques. The depth determination technique may be, e.g., direct time-of-flight (ToF) depth sensing, indirect ToF depth sensing, structured light, passive stereo analysis, active stereo analysis (uses texture added to the scene by light from the illuminator 140), some other technique to determine depth of a scene, or some combination thereof. In some embodiments, the imaging device 135 is oriented toward a mouth of the user, and the imaging device 140 may capture mouth related information (e.g., information about food being eaten), which can be utilized for, e.g., health-related diagnostic of the user wearing the headset 100.

The headset 100 includes various sensors embedded into the frame 105 for capturing data for a user wearing the headset 100. The sensors embedded into the frame 105 illustrated in FIG. 1 include at least one of: one or more eye sensors 115, a position sensor 130, a breath sensor 145, and an ambient light sensor 150. While FIG. 1 illustrates the sensors in example locations on the headset 100, the sensors may be located elsewhere on the headset 100. Similarly, there may be more or fewer sensors embedded into the frame 105 than what is shown in FIG. 1.

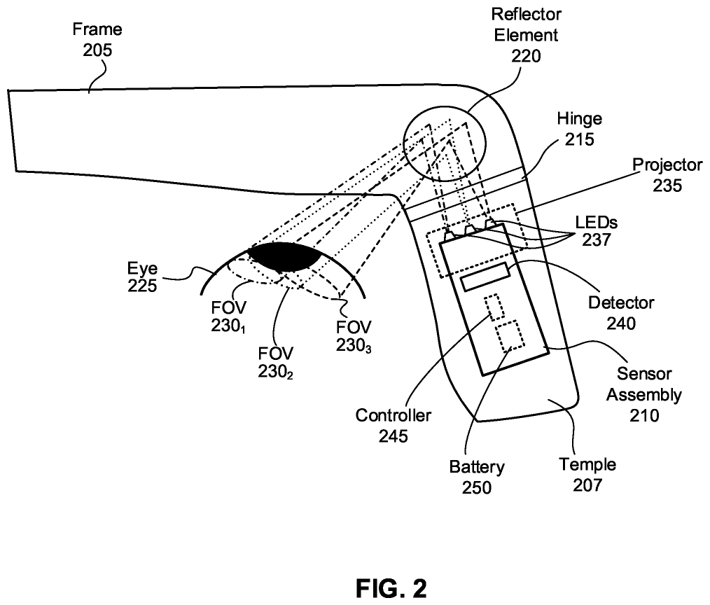

The eye sensor 115 may track a position of an eyelid of a user's eye over time and capture eyelid tracking information. The eye sensor 115 may capture the eyelid tracking information by, e.g., measuring an amount of occlusion over time of a pupil for the user's eye. The headset 100 may include a pair of eye sensors 115—one eye sensor 115 for each user's eye. The eye sensor 115 may be implemented as an eyelid tracking sensor that includes at least one light emission element and at least one photodiode. The eye sensor 115 may be part of a sensor assembly 125, and the sensor assembly 125 may further include the controller 120 and the power assembly 123. In one embodiment, the eye sensor 115 is implemented as an event sensor capturing information about “an event” (e.g., blink) occurred in relation to the user's eye. In another embodiment, the eye sensor 115 includes a single light emission diode (LED)/photodiode pair (i.e., pair of discrete components). In yet another embodiment, the eye sensor 115 is an off-the-shelf “proximity sensor” that modulates emitting light to reject interference with receiving light. In yet another embodiment, the eye sensor 115 comprises an array of LEDs/photodiodes, e.g., coupled with at least one optical elements (such as at least one cylindrical lens) to spread each LED/photodiode pair into an axis orthogonal to a blink direction axis. In yet another embodiment, the eye sensor 115 is an optical flow sensor that computes an optical flow in a field-of-view. In yet another embodiment, the eye sensor 115 is a complementary metal-oxide semiconductor (CMOS) imager for capturing a series of images from which eyelid tracking information can be deduced. More details about a structure of the eye sensor 115 are provided below in relation to FIG. 2.

The eyelid tracking information captured by the eye sensor 115 may be provided to the transceiver 127 to be further relayed to a secondary device (not shown in FIG. 1) coupled to the headset 100 for processing and determination of eyelid statistics. Alternatively, eyelid tracking information captured by the eye sensor 115 may be provided to the controller 120, and the controller 120 may process the captured eyelid tracking information and determine the eyelid statistics. The eyelid statistics may include, e.g., information about a PERCLOS (percentage of eyelid closure over the pupil) over time, a total blink duration, an eyelid closing duration, a hold duration at the “bottom” of the blink, an eyelid reopening duration, a speed of eyelid movement, some other eyelid statistics, or some combination thereof.

The eyelid statistics determined based on the eyelid tracking information captured by the eye sensor 115 can be indicative of psychomotor performance for the user, a sleep sensitivity for the user, a daily sleep need for the user, a sleep deprivation for the user, etc. The psychomotor performance for the user is a measure of the user's body reaction time, i.e., how long it takes for the user to see something, process it, and react accordingly. A reaction time may be estimated from a model that fits eyelid movement statistics to psychomotor vigilance test (PVT) performance. The model can be fit on a population level or tuned to an individual by a per-user calibration that can be performed once or be ongoing. The daily sleep need for the user can be defined as a number of hours that the user needs to sleep in order to have the psychomotor performance above a threshold level. The sleep sensitivity for the user is a measure of a time of sleep that the user can miss before it begins affecting the user's psychomotor performance the next day (e.g., when the psychomotor performance fall below a threshold level). The sleep deprivation can be defined as a number of hours accumulated over a defined time period that the user sleeps less than the user's average number of sleep hours.

In some embodiments, the eyelid statistics information for the user can be matched (e.g., at the secondary device or the controller 120) to a sleep deprivation model for a health-related diagnostic of the user (e.g., determination of user's psychomotor performance). The sleep deprivation model may be obtained by testing multiple subjects over time by collecting their sleep deprivation data. Sleep trackers may be worn by the test subjects that provide the sleep deprivation data, e.g., based on subjective inputs from the test subjects in relation to their tiredness over a defined period of time. The sleep deprivation data from the test subjects may be provided to the headset 100 and/or the secondary device as information about the sleep deprivation model, e.g., via one or more partner application devices of the test subjects communicatively coupled with the secondary device and/or the headset 100.

Sleep deprivation can be highly correlated with PVT performance. An estimate of psychomotor vigilance test performance (i.e., reaction time) that is derived from eyelid movement statistics may be index into a model that fits sleep deprivation to PVT performance. The PVT-sleep deprivation model can be fit to the population or calibrated individually per user, e.g., once or be continuously fit based on a feedback from the user.

While the eyelid statistics information can be used to measure sleep deprivation, the eyelid statistics information may also be used to estimate user's focus and/or attention—and thereby produce a mapping between amount of sleep deprivation and reduced focus. The mapping between amount of sleep deprivation and reduced focus can be useful in, e.g., providing the user with a qualitative measure of how much sleep they can lose before their work may start to suffer. For example, after getting a permission from an employee, an employer may issue the headset 100 to the employee and use the eyelid statistics information obtained at the headset 100 to track a fatigue metric vs. a psychomotor performance metric of the employee. If the psychomotor performance metric and/or the fatigue metric get above a threshold level, the employer may modify a shift schedule for the employee. Examples of professions that can utilize the eyelid statistics information for monitoring focus and/or attention of its employees may include: firemen, air traffic control personnel, pilots, professional drivers, medical professionals, or any other fields where fatigue of an employee could have major consequences.

Fatigue tracking measures through eyelid statistics (e.g., PERCLOS, blink duration statistics, etc.) can be used to determine various health-related metrics. For example, information about the eyelid statistics may be used to determine how long each individual user needs to sleep (e.g., an eight hour of sleep on average is an imprecise metric that does not apply to everyone), as well as the user's sleep sensitivity (i.e., how sensitive the user is to missing sleep). This can be estimated from eyelid statistics alone (e.g., captured by the one or more eye sensors 115) or in combination with sleep data gathered from other sleep tracking devices (e.g., wearable devices, sleep mats, etc.). Furthermore, the eyelid statistics may quantitatively measure a user's fatigue/psychomotor performance/energy state throughout the day. Additionally, or alternatively, the eyelid statistics may provide a measure on how a user's sleep needs change over time (e.g., daily, weekly, monthly) depending on various factors in their lives (e.g., are they sick, are they recently jet lagged, etc.). The eyelid statistics may be also utilized to correlate a user's sleep durations and user's sleep quality with their performance/energy levels throughout the day.

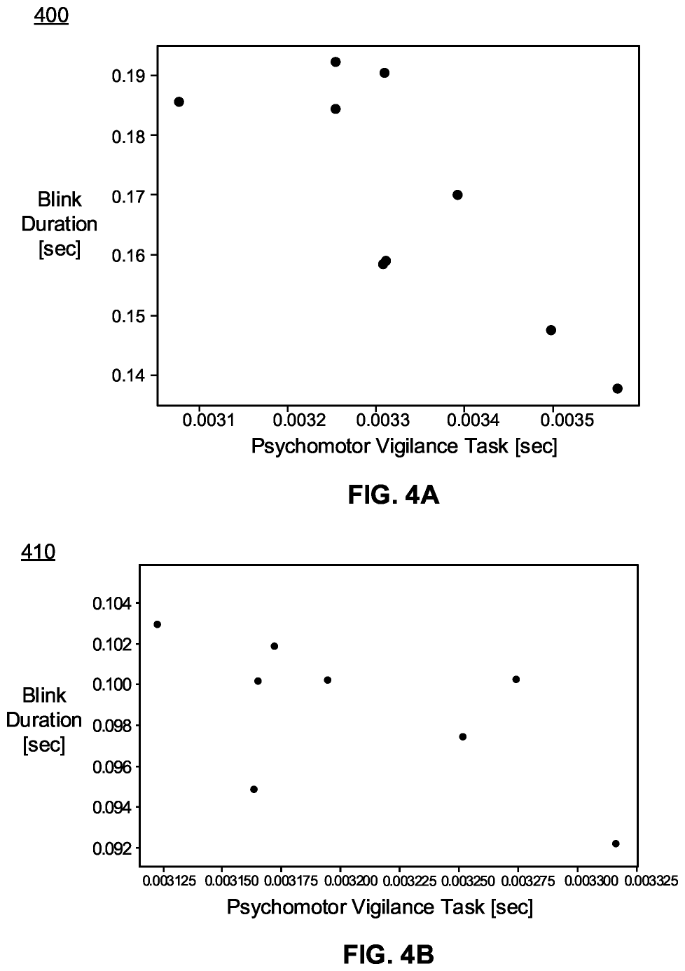

Eye blink duration statistics obtained from data captured by the one or more eye sensors 115 (e.g., time it takes for the eyelid to close, time that the eyelid is closed, and time it takes for the eyelid to open) can be used to estimate, e.g., psychomotor performance for the user. For example, the PVT is a sustained-attention reaction-timed task that measures a speed with which subjects respond to a visual or auditory stimulus. Reaction times and lapses in PVT experiments can be correlated to an increased fatigue and tiredness as well as a sleep debt (the amount of sleep required by the body subtracted by the amount of sleep received over the course of a defined time). The eye blink duration statistics may be correlated with PVT reaction times and lapses and can be used as a metric that is continuously monitored by the one or more eye sensors 115 measuring the eye and eyelid movements. In this manner, the eye blink duration statistics can be used to measure psychomotor performance for the user and correlate the measured psychomotor performance to sleep, track the psychomotor performance throughout the day, week, month, or year, and can be used to estimate the user's sleep need and sleep sensitivity. For example, daily PVT measurements/tracking can be used for suggesting changes to user's sleep habits and can be further integrated with direct methods of sleep tracking (e.g., sleep variables that measure user's time in bed). Hourly PVT measurements can be utilized for capturing variations in user's cognitive performance throughout the day and can be used for suggesting interventions to the user (e.g., naps or breaks). More details and examples of correlation between the eyelid statistics and psychomotor performance for the user are provided below in relation to FIGS. 4A through 7B.

The position sensor 130 generates one or more measurement signals in response to motion of the headset 100. The position sensor 130 may capture information about head orientation, head pose, head stability, user's posture, user's direction, etc., which can be utilized for, e.g., a health-related diagnostic of the user. Furthermore, the position sensor 130 may track information about user's steps and user's activity. The position sensor 130 may include an IMU. Examples of position sensor 130 include: one or more accelerometers, one or more gyroscopes, one or more magnetometers, another suitable type of sensor that detects motion, a type of sensor used for error correction of the IMU, or some combination thereof. The position sensor 130 may be located external to the IMU, internal to the IMU, or some combination thereof.

The breath sensor 145 may perform analysis of breath information gathered from the user, e.g., information about a level of CO₂ emitted by the user during breathing, humidity information (e.g., dehydration level) in a breath of the user, information about a level of alcohol in a breath of the user, a breath rate, some other breath information, or combination thereof. The breath information captured by the breath sensor 145 may be utilized (alone or in combination with other health information data captured by other sensors) for, e.g., a health-related diagnostic of the user. For example, a respiratory rate measured by the breath sensor 145 may be an early indicator of various physiological conditions such as hypoxia (low levels of oxygen in the cells), hypercapnia (high levels of carbon dioxide in the bloodstream), metabolic and respiratory acidosis, etc. Data captured by the breath sensor 145 can be processed at the headset 100, the secondary device, and/or the server platform.

A level of CO₂ may be measured using, e.g., a nondispersive infrared (NDIR) sensor or an electrochemical potentiometric gas sensor. An NDIR sensor may include an infrared source, a light tube, a bandpass filter, and a detector. A target gas a level of which is being measured may be determined through the selection of a filter wavelength. For measuring the level of CO₂, the filter wavelength may be, e.g., approximately 4.26 μm, representing a wavelength of light not being absorbed by other commonly found gases or by water vapor, which greatly reduces cross-sensitivities and impact to moisture and humidity. The normal operation of NDIR sensor may involve the gas being pumped or diffused into the light tube. The detector of the NDIR sensor may then measure the absorption of the characteristic wavelength of light. The amount of light absorption may be converted into an electrical output that provides a parts per million (ppm) measurement or a percentage of volume measurement. The more light being absorbed equates to more target gas molecules being present, which results in a lower output signal and inversely higher reported target gas (e.g., CO₂) concentration. On the other hand, an electrochemical potentiometric gas sensor may have a structure of an electrochemical cell, which consists of three functional components, e.g., a sensing electrode, a solid-state electrolyte, and a reference electrode. In such an arrangement, selectivity of the electrode materials can be used to detect gaseous species that is defining an electromotive force of the cell, measured as a cell potential.

The ambient light sensor 150 may capture information about a spectrum of visible light incident on the user's eye. The ambient light sensor 150 may include a visible light emitter for emitting visible light and a visible light detector (e.g., one or more photodiodes) capable of capturing information about intensity of visible light reflected from the pupil and/or one or more other surfaces of the user's eye. The spectrum of visible light incident on the user's eye may be related to a user's sleep and circadian rhythm. The spectrum information captured by the ambient light sensor 150 may be provided to the secondary device (e.g., via the transceiver 127) for processing and presentation to the user as part of user's sleep information, e.g., as an additional suggestion for the user to improve sleep habits. Alternatively, the spectrum information captured by the ambient light sensor 150 may be provided to the controller 120 that processes the captured spectrum information. Sleep information is information related to a user's sleep and user's performance in relation to the user's sleep. Sleep information may include, e.g., information about a daily sleep need for the user, information about a sleep deprivation for the user, information about a reaction time of the user for a particular sleep duration, information about a sleep excess for the user, information about a sleep sensitivity for the user, information about a psychomotor performance (e.g., psychomotor vigilance) for the user, some other information about the user's sleep or performance, or some combination thereof.

The controller 120 may control operations of one or more components of the headset 100. The controller 120 may be embedded into the frame 105 and coupled (i.e., interfaced) with the various sensors embedded into the frame 105, the imaging device 135, and the transceiver 127. The controller 120 may comprise a processor and a non-transitory computer-readable storage medium (e.g., memory). The controller 120 may be configured to obtain sensor data captured by the one or more sensors and process at least a portion of the captured sensor data. The controller 120 may store the sensor data on its own non-transitory storage medium. At a later time (e.g., during charging of the headset 100 and/or the secondary device), the controller 120 may provide the sensor data to the transceiver 127 for transmission to the secondary device. Alternatively, or additionally, the controller 120 can compress the sensor data to reduce a size of data being transferred to the secondary device, e.g., to fit data transfer into an available communication bandwidth.

In some embodiments, the controller 120 can extract one or more features related to the user from the captured sensor data. The extracted feature(s) may include one or more features of user's eyes, such as a blink type, blink rate, PERCLOS information, blink statistics (e.g., eyelid closing duration, duration of eyes being closed, eyelid opening duration, blink speed), some other eye feature, or combination thereof. The controller 120 may process the extracted feature(s) for performing, e.g., a health-related diagnostic of the user.

The transceiver 127 may communicate sensor data captured by various sensors of the headset 100 to a secondary device (e.g., a smartphone, laptop, tablet, personal computer, etc.) communicatively coupled to the headset 100. The transceiver 127 may communicate the sensor data to the secondary device continuously or intermittently. The transceiver 127 may be communicatively coupled to the secondary device via, e.g., a wired or wireless connection.

The power assembly 123 may provide power to various components of the headset. The power assembly 123 may comprise one or more rechargeable batteries. The power assembly 123 may provide power to, e.g., the eye sensor 115, the controller 120, the transceiver 127, the breath sensor 145, the ambient light sensor 150, the imaging device, and/or the illuminator 140. In one or more embodiments, the power assembly 123 is part of the sensor assembly 125 and provides power only to components of the sensor assembly 125.

The headset 100 described herein may be used for other applications uses in addition to those described above. Applications of the headset 100 can be in digital health, multisensory augmentation, augmented reality, virtual reality, mixed reality, fall detection, human-computer interaction, drowsiness detection (e.g., during driving), monitoring progression of neurological diseases, alerts/reminders (e.g., for prescriptions), cognitive load monitoring, stroke detection, some other application, or combination thereof.

FIG. 2 illustrates an example top view of a frame 205 of a headset, in accordance with one or more embodiments. The frame 205 may be an embodiment of the frame 105. The frame 205 may include a sensor assembly 210 and a reflector element 220. There may be more or fewer components on the frame 205 than what is shown in FIG. 2.

The sensor assembly 210 may track positions of an eyelid of an eye 225 of a user wearing the headset. Also, the sensor assembly 210 may capture eyelid tracking information. The sensor assembly 210 may be an embodiment of the sensor assembly 125. In one embodiment, as shown in FIG. 2, the sensor assembly 210 is embedded into a temple 207 of the frame 205, e.g., behind a hinge 215 of the frame 205. Note that when embedded into the temple 207 behind the hinge 215, the sensor assembly 210 does not require any wires to pass through the hinge 215 from the rest of electronics stored in the temple 207. In another embodiment, the sensor assembly 210 is clipped onto the temple 207. In yet another embodiment, the sensor assembly 210 is adhered to the temple 207. Alternatively, the sensor assembly 210 may positioned (e.g., embedded) into a front side of the frame 205. In such case, the sensor assembly 210 can be positioned to emit light directly at the eye 225 from the front side of the frame 205. The sensor assembly 210 may include a projector 235, a detector 240, a controller 245, and a battery 250. The sensor assembly 210 may include more or fewer components than what is shown in FIG. 2.

The projector 235 may emit light in accordance with emission instructions (e.g., from the controller 245). The emitted light may be projected toward the eye 225, e.g., directly and/or via the reflector element 220. The emitted light may be spherical light (i.e., light spread over a sphere or a portion of sphere in space), structured light, polarized light, IR light, some other type of light, or some combination thereof. The projector 235 may include at least one light emission element, e.g., at least one LED emitting light having a wavelength of 850 nm or 940 nm. In the embodiment illustrated in FIG. 2, the projector 235 includes an array of LEDs 237 (e.g., three LEDs). A light beam emitted from each LED 237 may have a respective path toward at least one surface (e.g., eyelid) of the eye 225, which is associated with a respective field-of-view (FOV). For example, as shown in FIG. 2, light beams emitted from the three LEDs 237 of the projector 235 may cover three different FOVs of the eye 225, e.g., FOVs 230₁, 230₂, 230₃.

The detector 240 may capture light originally emitted from the projector 235 (e.g., the array of LEDs 237) and reflected from the at least one surface (e.g., eyelid) of the eye 225. In embodiments when the emitted light is reflected from the eyelid of the eye 225, the detector 240 captures information about positions of the eyelid over time, i.e., the detector 240 captures eyelid tracking information. The detector 240 may include at least one photodiode (e.g., an array of photodiodes) configured to capture light reflected from the at least one surface of the eye 225. The at least one photodiode of the detector 240 may be configured as an IR photodiode.

The controller 245 may control operations of the projector 235 and the detector 240. The controller 245 may generate emission instructions (e.g., voltage signals) provided to one or more light emission elements of the projector 235. For example, the controller 245 may control emission operations of the LEDs 237 by providing a corresponding voltage signal to each LED 237. The controller 245 may further receive information about reflected light captured at the detector 240 over time, and determine eye tracking information (i.e., eyelid tracking information) using the received information about captured reflected light. In an embodiment, the controller 245 processes the eyelid tracking information to determine, e.g., various eyelid statistics. Alternatively, or additionally, the controller 245 may provide the eyelid tracking information to a transceiver (not shown in FIG. 2) for further communication to a secondary device coupled to the headset. In another embodiment, the detector 240 directly provides the captured eyelid tracking information to the transceiver for further communication to the secondary device.

The battery 250 may provide power to components of the sensor assembly 210, i.e., to the projector 235, the detector 240, and/or the controller 245. The battery 250 may be a rechargeable battery (e.g., lithium-based rechargeable battery) having “all day” battery life. Alternatively, the battery 250 may a replaceable non-rechargeable battery.

The reflector element 220 may reflect light emitted from the sensor assembly 210 (i.e., from the one or more light emission elements of the projector 235) towards an eye box of the eye 225. Additionally, the reflector element 220 may redirect light reflected from the eyelid towards the sensor assembly 210 (i.e., towards the detector 240). The reflector element 220 may be mounted on the frame 205, e.g., in front of the hinge 215, thus providing external light reflections (i.e., external relative to an outer surface of the frame 205). Alternatively, the reflector element 220 may be integrated into the frame 205, thus providing internal light reflections (i.e., internal relative to the outer surface of the frame 205). The reflector element 220 may operate as a spherical reflector, an IR reflector, some other type of reflector, or combination thereof. The reflector element 220 may be a mirror, a lens (e.g., with a partially reflective coating), a sphere reflector, a half sphere reflector, a parabolic reflector, a waveguide, “a birdbath optic” (i.e., a mirror combined with a beam splitter), some other optical element capable of reflecting incident light, or combination thereof. In one or more embodiments, the reflector element 220 is not required since the projector 235 is configured (e.g., by being appropriately positioned on the frame 205) to emit one or more light beams directly toward the eyelid of the eye 225. Also, in such an instance, light reflected from the eyelid of the eye 225 reaches the detector 240 without employment of the reflector element 220.

In embodiments where the reflector element 220 includes a waveguide, the waveguide of the reflector element 220 is configured to reflect light from the projector 235 to the eye 225, as well as to project (i.e., reflect) image light (content) to the eye 225 (e.g., image light generated by a display element in the lens 110). In some other embodiments where the reflector element 220 includes a birdbath optic, the birdbath optic of the reflector element 220 is configured to project light from the projector 235 to the eye 225, as well as to project (i.e., reflect) image light (content) to the eye 225 (e.g., image light emitted from a display element in the lens 110).

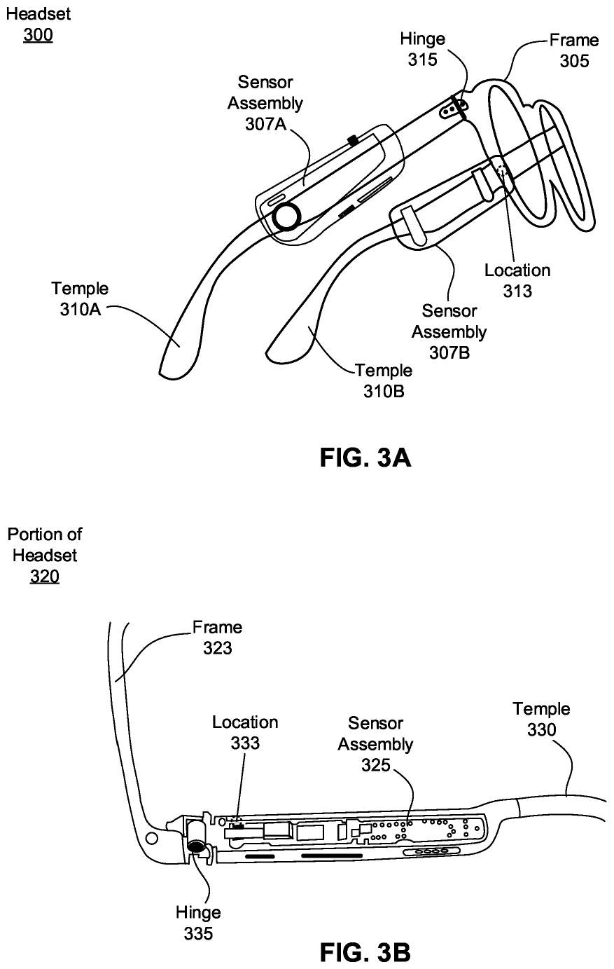

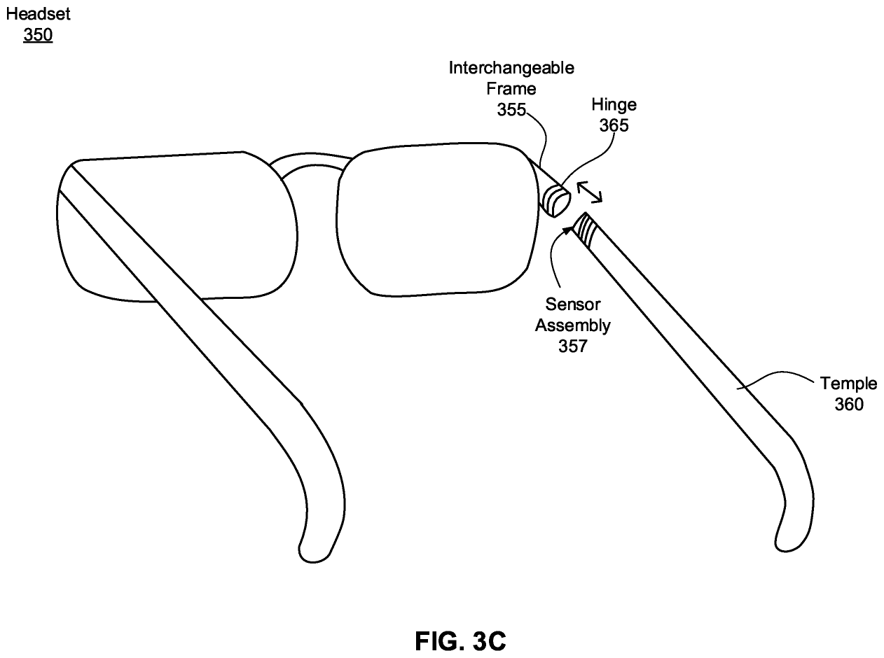

FIG. 3A illustrates an example headset 300 with sensor assemblies clipped onto temples of a frame 305 of the headset 300, in accordance with one or more embodiments. The headset 300 may be an embodiment of the headset 100. As shown in FIG. 3A, a sensor assembly 307A may be clipped onto a temple 310A of the frame 305, whereas a sensor assembly 307B may be clipped onto another temple 310B of the frame 305. The sensor assemblies 307A, 307B may be clipped onto respective temples 310A, 310B behind a hinge 315. Each sensor assemblies 307A, 307B may track an eyelid of a respective eye of a user wearing the headset 300 and capture eyelid tracking information for the eyelid of the respective eye. Light may shine directly from each sensor assembly 307A, 307B (e.g., from a respective location 313) to the respective eye of the user. The location 313 on the respective sensor assembly 307A, 307B may be as close to the hinge 315 as possible. Furthermore, light reflected from the respective eye of the user may be in-coupled to the respective sensor assembly 307A, 307B approximately at, e.g., the location 313. As the eyelid of the respective eye moves, the eyelid motion may appear as mainly vertical motion from the perspective of location 313 on the respective temple 310A, 310B. Each sensor assembly 307A, 307B can be unclipped from the respective temples 310A, 310B of the headset 300, and may be clipped onto temples of some other headset. Alternatively, each sensor assemblies 307A, 307B can adhere (permanently or temporarily) to the respective temples 310A, 310B. Each sensor assembly 307A, 307B may be an embodiment of the sensor assembly 210.

FIG. 3B illustrates an example portion of a headset 320 with a sensor assembly 325 embedded into a frame 323 of the headset 320, in accordance with one or more embodiments. The headset 320 may be an embodiment of the headset 100. The sensor assembly 325 may track an eyelid of an eye of a user wearing the headset 320 and capture eyelid tracking information for the eyelid. The sensor assembly 325 may be an embodiment of the sensor assembly 210. As shown in FIG. 3B, the sensor assembly 325 may be embedded into a temple 330 of the frame 323, e.g., behind a hinge 335. The sensor assembly 325 may be embedded into the frame 323 using an injection molding, overmolding, some other type of embedding process, or combination thereof. For the injection molding, the temple 330 may be injection molded in one or multiple pieces, such that a cavity in the temple 330 is made where the sensor assembly 325 could slide or be placed into. For the overmolding, the temple 330 may be directly injection molded around electronic components of the sensor assembly 325 creating a seamless temple. Light may shine directly from the sensor assembly 325 (e.g., from a location 333 on the temple 330) to the user's eye. The location 333 may be as close to the hinge 335 as possible. Additionally, light reflected from the user's eye may be in-coupled to the sensor assembly 325 approximately at, e.g., the location 333. As the eyelid of the user's eye moves, the eyelid motion may appear as mainly vertical motion from the perspective of location 333 on the temple 330.

FIG. 3C illustrates an example headset 350 with an interchangeable frame 355, in accordance with one or more embodiments. The interchangeable frame 355 includes a sensor assembly 357 embedded into a temple 360, e.g., behind a hinge 365. The headset 350 may be an embodiment of the headset 100, and the interchangeable frame 355 may be an embodiment of the frame 105. The sensor assembly 357 may track an eyelid of an eye of a user wearing the headset 350 and capture eyelid tracking information for the eyelid. The sensor assembly 357 may be an embodiment of the sensor assembly 210. In the configuration shown in FIG. 3C, at least a portion of the temple 360 behind the hinge 365 that includes the sensor assembly 357 can be removable from the frame 355 and can be attached to some other interchangeable frame.

Each sensor assembly presented herein (e.g., the sensor assembly 125, the sensor assembly 210, the sensor assemblies 307A-B, the sensor assembly 325, and the sensor assembly 357) may be primarily configured for detecting eyeblinks (i.e., movement of eyelids) and measuring durations of eyeblinks over time at a specific accuracy (e.g., millisecond precision, a detection rate above a detection threshold rate, and a rate of false positives below a false positives threshold rate) and in real-world scenarios (e.g., sunlight, head/frame movement or slippage while talking walking or adjusting frames, varying head and eye shapes or skin tones, no or a limited level of calibration). A blink duration is correlated with a PVT, which represents a common measure of reaction time, focused attention on a task, and overall fatigue. It is well known that the PVT is related (e.g., linearly) to a cumulative sleep debt. The cumulative sleep debt is a measure of the acute sleep deprivation, e.g., a number of hours of missed sleep from a user's individual baseline accumulated over a defined time period. Furthermore, it is well known that the acute PVT performance (e.g., PVT lapses) are correlated with a blink duration. Thus, it is expected that the cumulative sleep debt is directly correlated with a blink duration.

FIG. 4A illustrates an example graph 400 illustrating correlation between a blink duration and PVT performance for a first user (e.g., wearer of a headset), in accordance with one or more embodiments. FIG. 4B illustrates an example graph 410 illustrating correlation between a blink duration and a PVT performance for a second user (e.g., wearer of a headset), in accordance with one or more embodiments. It can be observed from the graphs 400, 410 that the PVT and blink duration are correlated as expected, i.e., a higher PVT is related to a shorter blink duration and a lower PVT is related to a longer blink duration. Just as acute sleep deprivation shows a correlation between rapidly declining PVT performance and blink duration, the graphs 400, 410 provides that chronic sleep changes incrementally affect average blink duration over time, by showing that the PVT and blink duration over multiple days and multiple users are correlated as expected. Therefore, by tracking eyelid positions and measuring blink durations over time (e.g., via the sensor assembly 125, the sensor assembly 210, the sensor assemblies 307A-B, the sensor assembly 325, and/or the sensor assembly 357), the user's PVT performance and user's psychomotor performance in general can be tracked and evaluated over time.

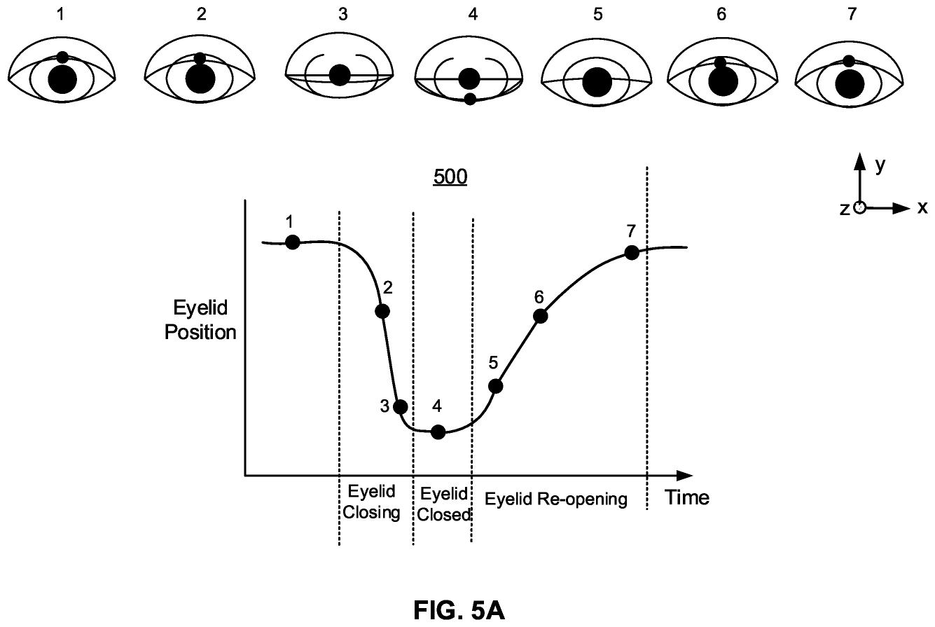

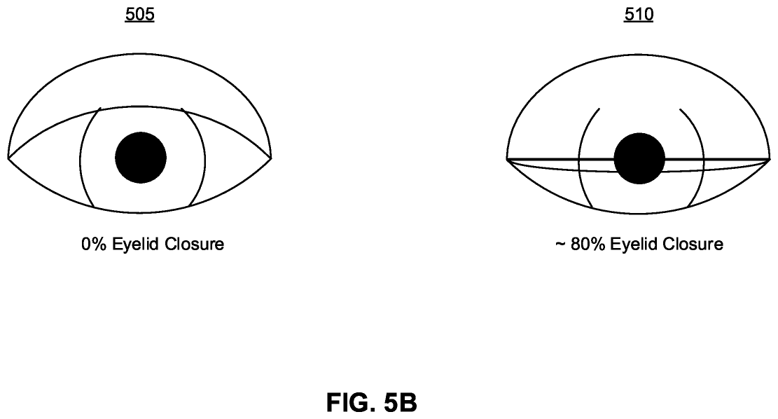

FIG. 5A illustrates an example eyelid tracking over time, in accordance with one or more embodiments. The eyelid tracking over time shown in FIG. 5A can be achieved by utilizing a sensor assembly mounted on a headset, e.g., the eye sensor 115, the sensor assembly 210, the sensor assemblies 307A-B, the sensor assembly 325, and/or the sensor assembly 357. The graph 500 in FIG. 5A shows various eyelid positions (e.g., along the y dimension) as a function of time. The graph 500 also illustrates that a blink duration has multiple subcomponents.

The eyelid closing and opening dynamics (i.e., blinking operation) is represented in FIG. 5A with seven stages. At stage 1, the eyelid covers, e.g., 20% of an eye (e.g., eye is 80% open), which can be defined as the eye “fully open” before the blink starts. At stage 2, the eyelid covers, e.g., 40% of the eye (e.g., eye is 60% open), which can be defined as a stage where the blink has already started. At stage 3, the eyelid covers, e.g., 80% of the eye (e.g., eye is 20% open), and at stage 4, the eyelid covers, e.g., 100% of the eye (e.g., eye is 0% open), which means that the eye is fully closed. At stage 5, the eyelid is in re-opening phase and covers, e.g., 60% of the eye (e.g., eye is 40% open). At stage 6, the eyelid continues to re-open and covers, e.g., 40% of the eye (e.g., eye is 60% open). Finally, at stage 7, the eyelid covers, e.g., 20% of the eye (e.g., eye is 80% open), which means that the eye is effectively “fully open” and the blink ends.

It can be observed from FIG. 5A that a first time duration covering the stages 2 and 3 can be defined as a “eyelid closing” time, a second time duration covering the stage 4 can be defined as a “eyelid closed” time, and a third time duration covering the stages 5, 6, and 7 can be defined as a “eyelid re-opening” time. In addition to these “time duration metrics”, some other blink metrics (or eyelid statistics) can be evaluated and correlated with the user's psychomotor performance. Example of some other blink metrics can include: a blink duration, PERCLOS, an eyelid closing speed, an eyelid reopening speed, a blink frequency, a blink interval, etc. These eyelid metrics can be determined at a secondary device communicatively coupled to the headset by processing eyelid tracking information captured at the headset.

FIG. 5B illustrates an example of eyelid metric (e.g., PERCLOS), in accordance with one or more embodiments. FIG. 5B illustrates an example 505 of PERCLOS equal to 0% that corresponds to un-occluded pupil (i.e., fully open eye), and an example 510 of PERCLOS equal to approximately 80% that corresponds to the pupil occluded by an eyelid at approximately 80% of a total pupil's front area. Information about PERCLOS over time is correlated to information on how long it takes for the user to blink. When the user gets more tired (e.g., lose more sleep over time), the user's psychomotor vigilance is getting slower and takes more time for the user to blink, which is manifested by an increase of PERCLOS over time.

As discussed above, the one or more eye sensors 115 of the headset 100 may capture eye data related to an amount of occlusion over time for the user's pupil—eyelid tracking information. The controller 120 may process eyelid tracking information captured by the one or more eye sensors 115 to obtain the eyelid statistics information represented by, e.g., one or more PERCLOS based parameters. Alternatively, the eyelid tracking information may be communicated from the headset 100 to the secondary device that processes the eyelid tracking information and obtains the one or more PERCLOS based parameters. An example of the PERCLOS based parameter may include an amount of time per minute that the PERCLOS is greater than a defined threshold percentage (e.g., 80% or 75%). Other examples of PERCLOS based parameters that can be determined at the secondary device by processing the eyelid tracking information may include, e.g., a speed of eyelid closure (e.g., an amount of time per minute it takes for PERCLOS to change from 0% to 80%), a speed of eyelid reopening (e.g., an amount of time per minute it takes for PERCLOS to change from 80% to 0%), an amount of time per minute the eyelid stay closed (e.g., an amount of time that the PERCLOS is at 100%), some other PERCLOS based parameter, or combination thereof.

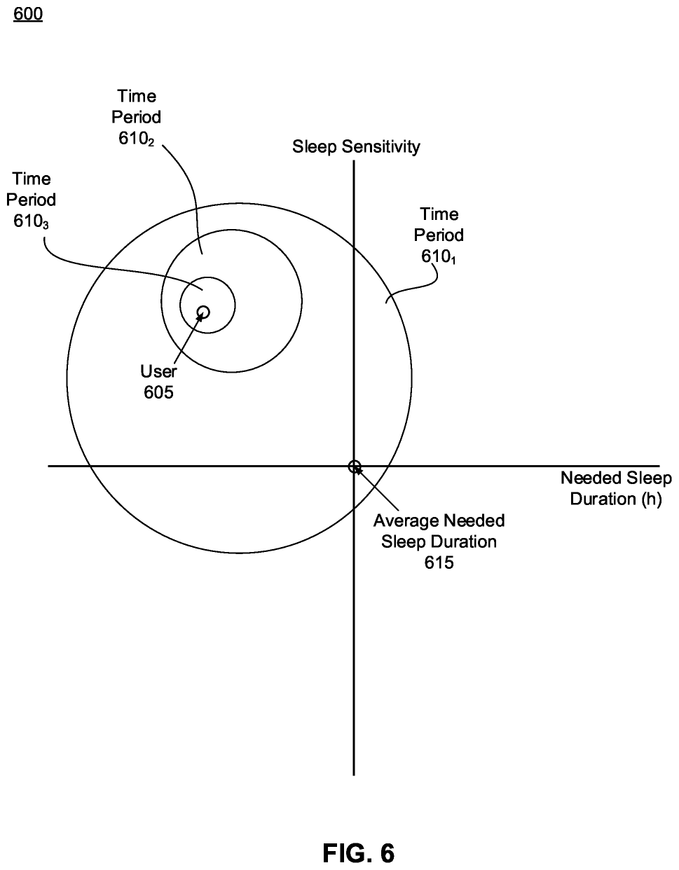

FIG. 6 illustrates an example graph 600 of a sleep sensitivity as a function of a needed sleep duration, in accordance with one or more embodiments. The sleep sensitivity is a measure of how a user is susceptible to losing psychomotor performance for a fixed amount of missed sleep. For example, a user with high sleep sensitivity may have a 20% drop in psychomotor performance after a night of sleep 1 hour less than the user's need, while another user with low sleep sensitivity may only exhibit a 5% drop in psychomotor performance for the same amount of missed sleep. It is known that the eight hours of recommended sleep do not generalize to every person. In fact, there is a very large distribution of sleep needs in the general population that changes with, e.g., age, medical conditions, illness, etc. After a few weeks (e.g., three weeks) of using the system presented herein, it would be possible to accurately estimate user's individual sleep parameters, such as a needed sleep duration and sleep sensitivity.

Sleep sensitivity data and needed sleep duration data shown in FIG. 6 may be determined, e.g., at a secondary device coupled to a headset (e.g., the headset 100). The sleep sensitivity data may be determined at the secondary device by correlating sleep data obtained from a sleep tracker (e.g., worn by the user) and eyelid tracking information (e.g., blink measurements) obtained from the headset. Similarly, the needed sleep duration data may be determined at the secondary device by combing the sleep data from the sleep tracker and eyelid statistics. The process illustrated in FIG. 6 may be performed at the secondary device by processing eyelid tracking information captured at the headset and the sleep data obtained from the sleep tracker worn by the user. Furthermore, the graph 600 may be shown to the user as part of a sleep app running on the secondary device.

Daily PVT performance may be a function of both daily sleep duration and fixed (or slowly varying) daily sleep need. By observing multiple data points of PVT performance and sleep durations over the course of multiple days, an estimate for the daily sleep need may begin to regress on the underlying function that relates the daily sleep need with PVT performance and sleep duration, considering that the sleep need is fixed or varying slower than the PVT performance and sleep duration. As more data points are gathered from the users, the estimates of daily sleep need may be refined continually. Sleep duration and PVT performance may be measured by a secondary device (e.g., smartwatch) and the eye-tracking-based measures described above, respectively. With a sufficiently accurate estimate on a user's sleep need, a secondary sleep duration measurement device may be eliminated, and the sleep duration may be estimated based on ongoing measurements of PVT performance and a priori known sleep need.

As shown in FIG. 6, a first range of sleep sensitivity for a first range of needed sleep duration for a specific user 605 may be determined after a first time period 610₁ (e.g., after one week). Then, a second range of sleep sensitivity (smaller than the first range of sleep sensitivity) for a second range of needed sleep duration (smaller than the first range of needed sleep duration) may be determined after a second time period 610₂ (e.g., after cumulative two weeks) longer than the first time period 610₁. After that, a third range of sleep sensitivity (smaller than the second range of sleep sensitivity) for a third range of needed sleep duration (smaller than the second range of needed sleep duration) may be determined after a third time period 610₃ (e.g., after cumulative three weeks) longer than the second time period 610₂. This process can continue until a range of sleep sensitivity is smaller than a first threshold range and a range of needed sleep duration is smaller than a second threshold range. Then, a sleep sensitivity and a needed sleep duration for the specific user 605 may be determined with a predefined accuracy. It can be observed from the graph 600 that the needed sleep duration for the user 605 is substantially different (i.e., shorter) than an “average needed sleep duration” 615 (e.g., of 8 hours). Based on information in the graph 600 provided to the user (e.g., as part of the sleep app running on the secondary device), the user may adjust his/her own sleep duration over time.

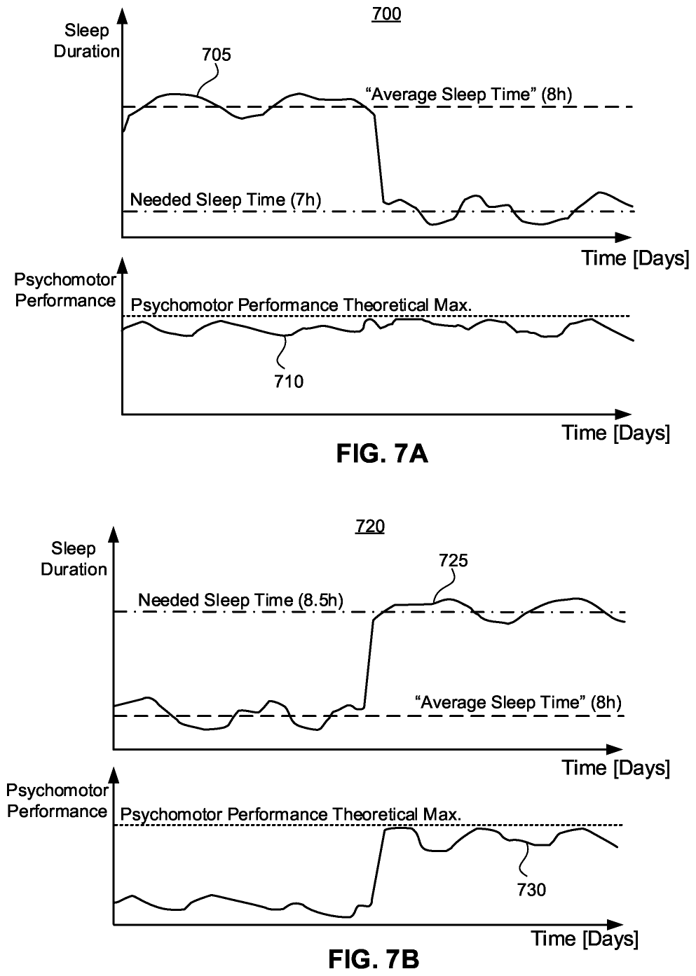

FIG. 7A illustrates an example graph 700 showing psychomotor performance correlated with a sleep duration for a first user, in accordance with one or more embodiments. A sleep duration plot 705 shows that the first user initially sleeps around the “average sleep time” (e.g., 8 hours), while the first user's needed sleep time has been evaluated at a sleep duration below the “average sleep time”, e.g., at 7 hours. This means that the first user can sleep less (e.g., one hour less, as shown by the later stage of the sleep duration plot 705), while maintaining the same psychomotor performance (as shown by a psychomotor performance plot 710).

FIG. 7B illustrates an example graph 720 showing psychomotor performance correlated with a sleep duration for a second user, in accordance with one or more embodiments. A sleep duration plot 725 shows that the second user initially sleeps around the “average sleep time” (e.g., 8 hours), while the second user's needed sleep time has been evaluated at a sleep duration above the “average sleep time”, e.g., at 8.5 hours. It can be also observed from a psychomotor performance plot 730 that psychomotor performance for the second user are not close enough to a theoretical maximum level when the second user sleeps around the “average sleep time.” Thus, even though the second user was sleeping for the “average sleep time” per night (e.g., 8 hours), the second user required a longer sleep duration (e.g., 8.5 hours), and thus the second user was never hitting the second user's peak psychomotor performance. This means that the second user should sleep more (e.g., approximately a half an hour more, as shown by the later stage of the sleep duration plot 725) to hit the peak psychomotor performance. In such an instance, as shown by the later stage of the psychomotor performance plot 730, the second user's psychomotor performance are getting closer to the theoretical maximum level. Note that the sleep statistics and psychomotor performance shown in FIGS. 7A-7B may be determined and evaluated at a secondary device coupled to a headset (e.g., the headset 100) by processing eyelid tracking information captured at the headset. Furthermore, the graphs 700, 720 may be shown to the user as part of a sleep app running on the secondary device. The graphs 700, 720 may be utilized by the user for e.g., establishing a baseline level of sleep for good psychomotor performance.

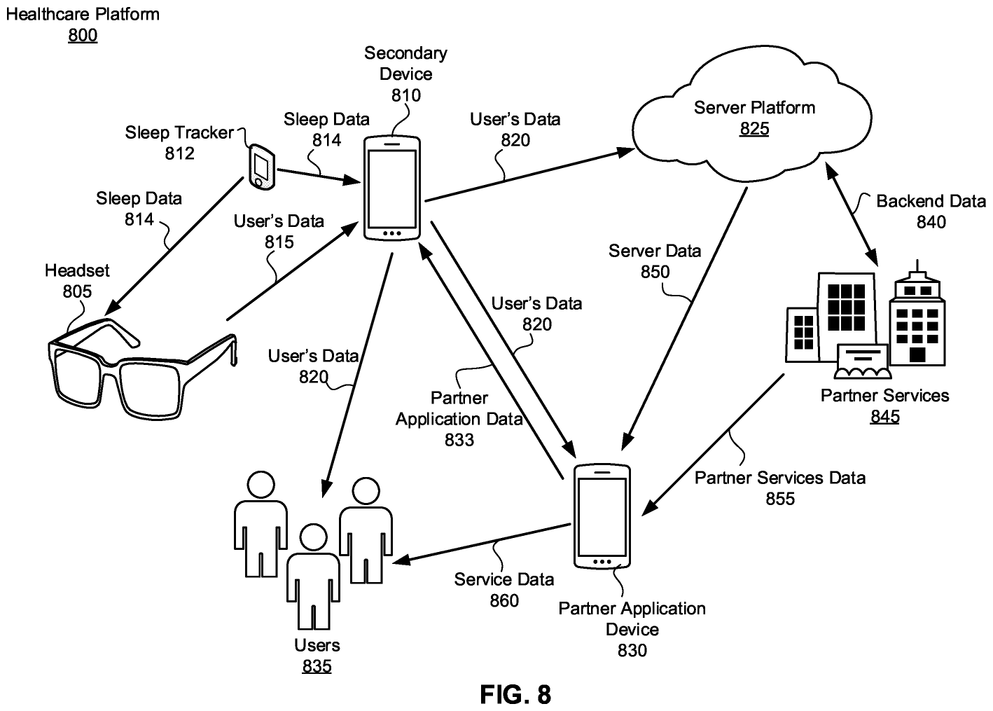

FIG. 8 illustrates an example healthcare platform 800 with a headset 805, in accordance with one or more embodiments. The headset 805 may be an embodiment of the headset 100. The headset 805 (e.g., electronic eyeglasses) as part of the healthcare platform 800 may capture user's data 815 (e.g., eyelid tracking information) via one or more sensors mounted on the headset 805 (not shown in FIG. 8). The one or more sensors of the headset 805 may be embodiments of the one or more eye sensors 115, the sensor assembly 125, the position sensor 130, the breath sensor 145 and/or the ambient light sensor 150. The headset 805 can be interfaced (e.g., via a wired or wireless connection) with a secondary device 810. In addition to the headset 805 and the secondary device 810, the healthcare platform 800 may include a sleep tracker 812, a server platform 825, one or more partner application devices 830, and one or more partner services 845. There may be more or fewer components of the than healthcare platform 800 what is shown in FIG. 8.

The secondary device 810 can be, e.g., a smartphone, laptop, desktop computer, tablet, a VR system, an AR system, a MR system, some other device or system, or combination thereof. The headset 805 may communicate the captured user's data 815 to the secondary device 810, e.g., via a wired or wireless connection. The user's data 815 may include raw data captured at the headset 805 and/or information about one or more features (e.g., eyelid statistics) extracted from the user's raw data. The user's data 815 may include eyelid tracking information captured by one or more sensors of the headset 805. The wired connection between the headset 805 and the secondary device 810 may be implemented as, e.g., a security digital (SD) card connection, Universal Serial Bus (USB) connection, Ethernet connection, some other wired connection, or combination thereof. The wireless connection between the headset 805 and the secondary device 810 may be implemented as, e.g., a Bluetooth, WiFi, some other wireless connection, or combination thereof. In one embodiment, the user's data 815 can be transferred from the headset 805 to the secondary device 810 in batches, i.e., as offline offloading of data. In another embodiment, the user's data 815 can be transferred continuously from the headset 805 to the secondary device 810.

Some portion of the user's data 815 occupying a higher portion of an available communication bandwidth (e.g., full raw image data) can be communicated to the secondary device 810 at a frequency lower than a threshold frequency (i.e., at a low frequency). In some other embodiments, some other portion of the user's data 815 occupying a lower portion of the available communication bandwidth (e.g., basic eyelid tracking information such as pupil occlusion data) can be communicated to the secondary device 810 at a frequency higher than the threshold frequency (e.g., at a high frequency).

The secondary device 810 may perform (e.g., via a controller of the secondary device 810) processing of the captured raw user's data 815 obtained from the headset 805. The secondary device 810 may also extract one or more features (e.g., eyelid statistics) from the user's data 815. In some embodiments, the secondary device 810 may perform processing of high resolution user's data (e.g., full image data) at a frequency lower than a threshold frequency (i.e., at a low frequency, such as once a day). In some other embodiments, e.g., to obtain information about trends, the secondary device 810 may perform processing of intermediate data results (i.e., user's data previously pre-processed at the headset 805) at a frequency higher than the threshold frequency (i.e., at a mid-frequency, such as several times per hour). In some other embodiments, the secondary device 810 may perform processing of raw user's data (e.g., eyelid position data) at a frequency higher than another threshold frequency (i.e., at a high frequency).

The secondary device 810 may provide user's data 820 to a server platform 825 (e.g., cloud platform) and/or at least one third party application device, i.e., the partner application device(s) 830. The user's data 820 may comprise a portion of the raw user's data 815 and another portion of processed user's data. Alternatively, or additionally, the user's data 820 can be utilized by one or more users 835 of the secondary device 810. Furthermore, one or more specific health-related applications can be deployed on the secondary device 810, e.g., to utilize the user's data 815 transferred from the headset 805.

The secondary device 810 may use information about pupil's occlusion captured at the headset 805 (i.e., eyelid tracking information) to determine various eyelid statistics information for the user. Furthermore, the secondary device 810 may correlate the determined eyelid statistics information to a sleep deprivation model of multiple test subjects for a health-related diagnostic of the user (e.g., determination of user's psychomotor performance, user's sleep sensitivity, user's daily sleep need, user's sleep deprivation, etc.). The secondary device 810 may obtain information about the sleep deprivation model from, e.g., the one or more partner application devices 830 (e.g., one partner application device 830 for each test subject) as part of partner application data 833 transmitted (e.g., via a wireless link) from the one or more partner application devices 830 to the secondary device 810 and/or the sleep tracker 812.

User's psychomotor performance can change day to day. For example, user's individual sleep requirements may change if the user is sick, or jet lagged. The secondary device 810 may accurately measure these daily changes in psychomotor performance (e.g., using the user's data 815) and inform the user on know how much sleep the user should be targeting to maintain a specific level of psychomotor performance. Additionally, or alternatively, the user's psychomotor performance can change hour by hour. The secondary device 810 may estimate the user's own daily circadian rhythm (e.g., using the user's data) and learn, e.g., how caffeine, meditation, meetings, etc. affect the user's own rhythm and energy levels throughout the day.

The secondary device 810 may be communicatively coupled (e.g., via a wired or wireless connection) with the sleep tracker 812. Additionally or alternatively, the sleep tracker 812 may be communicatively coupled (e.g., via a wired or wireless connection) to the headset 805. The sleep tracker 812 may be a wearable device (e.g., smartwatch, fitness tracker device, etc.) worn by the user that is capable of collecting sleep data 814 for the user. The sleep data 814 may include, e.g., information about sleep duration as a function of time, information about sleep deprivation as a function of time, information about sleep excess as a function of time, some other data related to the user's sleep habit, or some combination thereof. The sleep tracker 810 may provide the sleep data 814 to the headset 805 and/or the secondary device 810. The secondary device 810 (and/or the headset 805) may combine a processed version of the user's data 815 (e.g., processed eyelid tracking information) with the sleep data 814 from the sleep tracker 812 to determine sleep information for the user.

The secondary device 810 may serve as a relay node for transferring the user's data 815 from the headset 805 to the server platform 825. Data from the secondary device 810 (e.g., raw data, extracted user's features, determined user's statistics, some other user's data, or combination thereof, collectively referred to as the user's data 820) can be transferred (e.g., uploaded) to the server platform 825, e.g., by a transceiver or some other communication module of the secondary device 810. In some embodiments, the user may adjust privacy settings to allow or prevent the secondary device 810 from providing the user's data 820 to any remote systems including the server platform 825.

The server platform 825 can perform advance processing on the user's data 820 received from the secondary device 810. In some embodiments, the server platform 825 can perform high compute image processing on full raw image data captured (e.g., at a low frequency) by one or more imaging devices mounted on the headset 805. In some other embodiments, the server platform 825 can perform advanced processing on the raw user's data and/or compressed user's data (or features) uploaded from the secondary device 810.

In some embodiments, the server platform 825 can provide user's data (e.g., with or without advance processing being applied on the user's data) as backend data 840 to the one or more partner services 845 (e.g., partner server platforms or partner cloud services), e.g., via one or more backend communication channels between the server platform 825 and the one or more partner services 845. The server platform 825 may operate as a node that one or more external parties (i.e., the one or more partner services 845) can connect to and access the user's data through, e.g., an API of the server platform 825.

Various health related applications can be built on top of the API of the server platform 825 for several different purposes. At least some of the health related applications can be built for utilization by one or more external third parties (e.g., the one or more partner application devices 830). Alternatively, or additionally, one or more health related applications can be built internally, e.g., for utilization by the secondary device 810. To implement their own algorithms, the one or more external parties (e.g., the one or more partner application devices 830) may require access to the user's data that the server platform 825 can provide, e.g., as server data 850. Alternatively, the user's data 820 can be directly provided to the one or more partner application devices 830 from the secondary device 810. For example, the one or more other external parties (e.g., the one or more partner application devices 830) may only require access to features extracted from the raw user's data 815 (e.g., extracted at the secondary device 810 or at the server platform 825) for ease of development. The server platform 825 may offer functions that expose individual data streams at a particular time instant, or during a time series. The server platform 825 may apply different levels of processing (e.g., high frequency processing, mid-frequency frequency, low frequency processing, etc.) on the user's data 820 acquired from the secondary device 810 to provide various statistics on changes in certain data features, e.g., over the course of the minute, hour, day, week, etc.

In some embodiments, upon a request from the partner application device 830, the server platform 825 can provide raw user's data (e.g., raw data captured by one or more sensors mounted on the headset 805) and/or output data (e.g., user's data processed at the secondary device 810) as the server data 850 to the partner application device 830, e.g., via the API of the server platform 825. Similarly, as for the implementation of secondary device 810, the partner application device 830 can be implemented as, e.g., a smartphone, laptop, desktop computer, tablet, AR system, VR system, MR system, some other device or system, or combination thereof. Furthermore, the one or more partner services 845 (i.e., partner server platforms) can provide some user's data (e.g., mobile health data) as partner services data 855 to the partner application device 830.

In some embodiments, the partner services data 855 communicated from the one or more partner services 845 to the partner application device 830 are high compute low frequency services (e.g., full resolution image data) obtained through high compute processing at the server platform 825 or at the one or more partner server platforms of the one or more partner services 845. In some other embodiments, the partner services data 855 communicated from the one or more partner services 845 to the partner application device 830 are mid-compute high frequency services that can be further processed at the partner application device 830. Examples of the mid-compute high frequency services include but are not limited to pattern recognition and/or filtering of stored user's data over time to detect subtle changes in diagnostic properties of the user's data. In some other embodiments, the partner application device 830 can directly obtain at least a portion of the user's data 820 from the secondary device 810, which can be further processed and utilized by the partner application device 830. The one or more users 835 can utilize service data 860 with one or more partner services running on the partner application device 830.

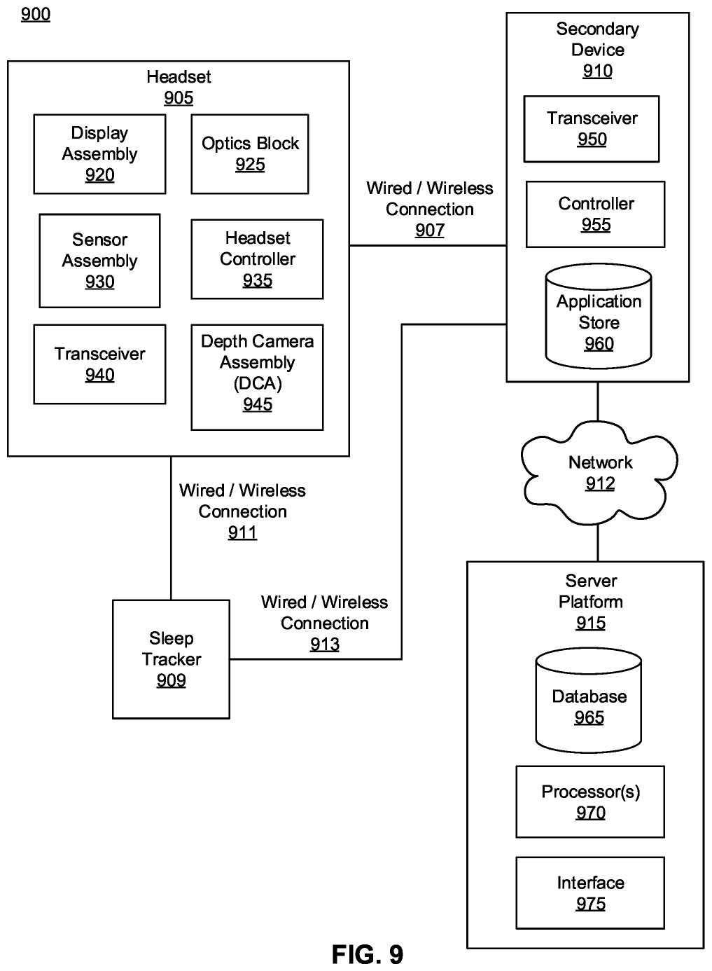

FIG. 9 is a block diagram of a healthcare platform 900 that includes a headset 905, in accordance with one or more embodiments. The healthcare platform 900 shown by FIG. 9 includes the headset 905, a secondary device 910, and a server platform 915 coupled to the secondary device 910 via a network 912. Additionally, the healthcare platform 900 may include a sleep tracker 909 coupled to the headset 905 and/or the secondary device 910. In some embodiments, the healthcare platform 900 may be the healthcare platform 800, the headset 905 may be the headset 100 or the headset 805, the secondary device 910 may be the secondary device 810, and the server platform 915 may be the server platform 825. In alternative configurations, different and/or additional components may be included in the healthcare platform 900. Additionally, functionality described in conjunction with one or more of the components shown in FIG. 9 may be distributed among the components in a different manner than described in conjunction with FIG. 9 in some embodiments.

The headset 905 includes a display assembly 920, an optics block 925, a sensor assembly 930, a headset controller 935, a transceiver 940, and a DCA 945. Some embodiments of the headset 905 have different components than those described in conjunction with FIG. 9. Additionally, the functionality provided by various components described in conjunction with FIG. 9 may be differently distributed among the components of the headset 905 in other embodiments or be captured in separate assemblies remote from the headset 905.

The display assembly 920 displays content to a user wearing the headset. The display assembly 920 displays the content using one or more display elements (e.g., the lenses 110). A display element may be, e.g., an electronic display. In various embodiments, the display assembly 920 comprises a single display element or multiple display elements (e.g., a display for each eye of the user). Examples of an electronic display include: a liquid crystal display (LCD), an organic light emitting diode (OLED) display, an active-matrix organic light-emitting diode display (AMOLED), a waveguide display, some other display, or some combination thereof. In some embodiments, the display assembly 920 includes some or all of the functionality of the optics block 925.

The optics block 925 may magnify image light received from the electronic display, corrects optical errors associated with the image light, and presents the corrected image light to one or both eye boxes of the headset 905. In various embodiments, the optics block 925 includes one or more optical elements. Example optical elements included in the optics block 925 include: an aperture, a Fresnel lens, a convex lens, a concave lens, a filter, a reflecting surface, a waveguide, a birdbath optic, or any other suitable optical element that affects image light. Moreover, the optics block 925 may include combinations of different optical elements. In some embodiments, one or more of the optical elements in the optics block 925 may have one or more coatings, such as partially reflective or anti-reflective coatings.

Magnification and focusing of the image light by the optics block 925 allows the electronic display to be physically smaller, weigh less, and consume less power than larger displays. Additionally, magnification may increase the field of view of the content presented by the electronic display. For example, the field of view of the displayed content is such that the displayed content is presented using almost all (e.g., approximately 110° diagonal), and in some cases, all of the user's field of view. Additionally, in some embodiments, the amount of magnification may be adjusted by adding or removing optical elements.

In some embodiments, the optics block 925 may be designed to correct one or more types of optical error. Examples of optical error include barrel or pincushion distortion, longitudinal chromatic aberrations, or transverse chromatic aberrations. Other types of optical errors may further include spherical aberrations, chromatic aberrations, or errors due to the lens field curvature, astigmatisms, or any other type of optical error. In some embodiments, content provided to the electronic display for display is pre-distorted, and the optics block 925 corrects the distortion when it receives image light from the electronic display generated based on the content.

The sensor assembly 930 may capture data related a user wearing the headset 905. In some embodiments, the sensor assembly 930 may include at least one of the one or more eye sensors 115, the position sensor 130, the breath sensor 145, and the ambient light sensor 150. Alternatively, the sensor assembly 930 may be configured to perform the same operations as at least one of the one or more eye sensors 115, the position sensor 130, the breath sensor 145, and the ambient light sensor 150. The sensor assembly 930 may be an embodiment of the sensor assembly 125 or the sensor assembly 210.

The headset controller 935 may process at least a portion of the user's data captured by the sensor assembly 930 and provide the processed user's data to the transceiver 940. In some embodiments, the headset controller 935 may be the controller 120 or configured to perform the same operations as the controller 120.

The transceiver 940 may communicate, via the wired or wireless connection 907, the user's data captured by the sensor assembly 930 to the secondary device 910 for processing of the captured user's data and utilization of the processed user's data for, e.g., a health-related diagnostic of the user. In some embodiments, the transceiver 940 may be the transceiver 127 or configured to perform the same operations as the transceiver 127.

The DCA 945 generates depth information for a portion of a local area of the headset 905. The DCA 945 includes one or more imaging devices and a DCA controller. The DCA 945 may also include an illuminator. Operation and structure of the DCA 945 is described above in conjunction with FIG. 1.

The wired connection 907 between the headset 905 and the secondary device 910 may be implemented as, e.g., a SD card connection, USB connection, Ethernet connection, some other wired connection, or combination thereof. The wireless connection between the headset 905 and the secondary device 910 may be implemented as, e.g., a Bluetooth, WiFi, some other wireless connection, or combination thereof.

The secondary device 910 may be, e.g., a smartphone, laptop, desktop computer, tablet, a VR system, an AR system, a MR system, some other device or system, or combination thereof. The secondary device 910 includes a transceiver 950, a controller 955, and an application store 960. Some embodiments of the secondary device 910 may have different components than those described in conjunction with FIG. 9. Additionally, the functionality provided by various components described in conjunction with FIG. 9 may be differently distributed among the components of the secondary device 910 in other embodiments or be captured in separate assemblies remote from the secondary device 910.

The transceiver 950 may receive the user's data from the headset 905. The transceiver 950 may also transfer (e.g., upload via the network 912) the received user's data and/or a processed version of the received user's data to the server platform 915. The transceiver 950 may further transmit the received user's data and/or the processed version of received user's data to one or more partner application devices (not shown in FIG. 9).

The controller 955 may perform processing of the user's data obtained from the headset 905. The controller 955 may also determine one or more features (e.g., eyelid statistics) from the raw user's data. The controller 955 may further perform processing of high resolution user's data (e.g., full image data). In some embodiments, the controller 955 may perform processing of intermediate data results (i.e., user's data previously pre-processed at the headset 905).

The application store 960 stores one or more health-related applications for execution at the secondary device 910 (e.g., by the controller 955). An application is a group of instructions, that when executed by the controller 955, generates content for presentation to the user. Content generated by an application may be in response to inputs received from the user. Examples of health-related applications include: an application for a health-related diagnostic based on information about user's eyelid statistics over time, detection of the user's activity for a period of time, an application for a health-related diagnostic based on user's breathing, posture monitoring, or other suitable health-related applications.

The sleep tracker 909 may be a wearable device (e.g., smartwatch, fitness tracker device, etc.) worn by the user. The sleep tracker 909 may collect sleep data for the user wearing the headset 905 (and/or one or more other users). The sleep data collected by the sleep tracker 909 may include, e.g., information about sleep duration as a function of time, information about sleep deprivation as a function of time, information about sleep excess as a function of time, some other data related to the user's sleep habit, or some combination thereof. The sleep tracker 909 may provide the sleep data to the headset 905 (e.g., via a wired or wireless connection 911) and/or the secondary device 910 (e.g., via a wired or wireless connection 913). The secondary device 910 (and/or the headset 905) may combine eyelid tracking information (e.g., after being processed to determine eyelid statistics or blink metrics) with the sleep data from the sleep tracker 909 to determine sleep information for the user. The sleep tracker 909 may be an embodiment of the sleep tracker 812.

The network 912 couples the secondary device to the server platform 915. The network 912 may include any combination of local area and/or wide area networks using both wireless and/or wired communication systems. For example, the network 912 may include the Internet, as well as mobile telephone networks. In one embodiment, the network 912 uses standard communications technologies and/or protocols. Hence, the network 912 may include links using technologies such as Ethernet, 802.11, worldwide interoperability for microwave access (WiMAX), 2G/3G/4G mobile communications protocols, digital subscriber line (DSL), asynchronous transfer mode (ATM), InfiniBand, PCI Express Advanced Switching, etc. Similarly, the networking protocols used on the network 912 can include multiprotocol label switching (MPLS), the transmission control protocol/Internet protocol (TCP/IP), the User Datagram Protocol (UDP), the hypertext transport protocol (HTTP), the simple mail transfer protocol (SMTP), the file transfer protocol (FTP), etc. The data exchanged over the network 912 can be represented using technologies and/or formats including image data in binary form (e.g. Portable Network Graphics (PNG)), hypertext markup language (HTML), extensible markup language (XML), etc. In addition, all or some of links can be encrypted using conventional encryption technologies such as secure sockets layer (SSL), transport layer security (TLS), virtual private networks (VPNs), Internet Protocol security (IPsec), etc.

The server platform 915 includes a database 965, one or more processors 970, and an interface 975. Some embodiments of the server platform 915 have different components than those described in conjunction with FIG. 9. Additionally, the functionality provided by various components described in conjunction with FIG. 9 may be differently distributed among the components of the server platform 915 in other embodiments or be captured in separate assemblies remote from the server platform 915.

The database 965 may store user's data (e.g., raw user's data as captured by the sensor assembly 930 and/or the processed version of user's data as processed at the secondary device 910). The database 965 may be a non-transitory computer readable storage medium.

The one or more processors 970 may efficiently perform a large number of computations to, e.g., extract various statistics and/or features from the user's data obtained from the secondary device 910 for exposing the extracted data to third parties through, e.g., the interface 975. The one or more processors 970 may also perform advance processing on the user's data obtained from the secondary device 910 (e.g., high compute image processing). Further, the one or more processors 970 may apply different levels of processing (e.g., high frequency processing, mid-frequency frequency, low frequency processing, etc.) on the user's data acquired from the secondary device 910 to provide various statistics on changes in certain data features.

The interface 975 may connect the server platform 915 with one or more partner server platforms (not shown in FIG. 9) and/or the one or more partner application devices for transferring the user's health data (e.g., as processed by the one or more processors 970). In some embodiments, the interface 975 may be implemented as an API. The API of the server platform 915 may be implemented using one or more programming languages, e.g., Python, C, C++, Swift, some other programming language, or combination thereof.

One or more components of the healthcare platform 900 may contain a privacy module that stores one or more privacy settings for user data elements. The user data elements describe the user, the headset 905 or the secondary device 910. For example, the user data elements may describe sensitive health information data of the user, a physical characteristic of the user, an action performed by the user, a location of the user of the headset 905, a location of the headset 905, a location of the secondary device 910, etc. Privacy settings (or “access settings”) for a user data element may be stored in any suitable manner, such as, for example, in association with the user data element, in an index on an authorization server, in another suitable manner, or any suitable combination thereof.

A privacy setting for a user data element specifies how the user data element (or particular information associated with the user data element) can be accessed, stored, or otherwise used (e.g., viewed, shared, modified, copied, executed, surfaced, or identified). In some embodiments, the privacy settings for a user data element may specify a “blocked list” of entities that may not access certain information associated with the user data element. The privacy settings associated with the user data element may specify any suitable granularity of permitted access or denial of access. For example, some entities may have permission to see that a specific user data element exists, some entities may have permission to view the content of the specific user data element, and some entities may have permission to modify the specific user data element. The privacy settings may allow the user to allow other entities to access or store user data elements for a finite period of time.

The healthcare platform 900 may include one or more authorization/privacy servers for enforcing privacy settings. A request from an entity for a particular user data element may identify the entity associated with the request and the user data element may be sent only to the entity if the authorization server determines that the entity is authorized to access the user data element based on the privacy settings associated with the user data element. If the requesting entity is not authorized to access the user data element, the authorization server may prevent the requested user data element from being retrieved or may prevent the requested user data element from being sent to the entity. Although this disclosure describes enforcing privacy settings in a particular manner, this disclosure contemplates enforcing privacy settings in any suitable manner.

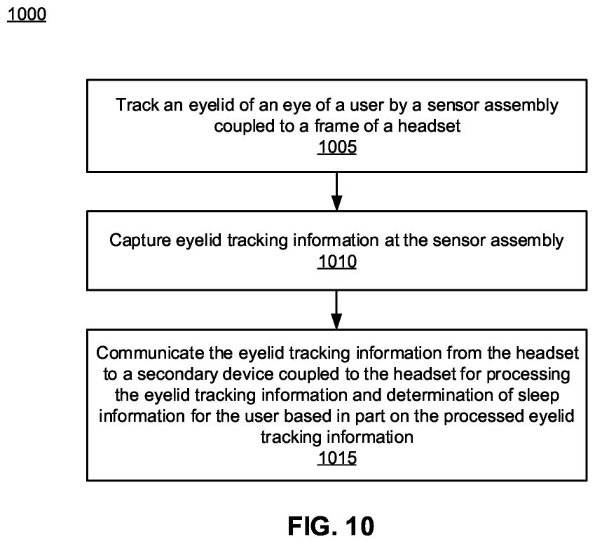

FIG. 10 is a flow chart illustrating a process 1000 performed at a headset for capturing eyelid tracking information used for evaluating psychomotor performance of a user of the headset, in accordance with one or more embodiments. The process 1000 of FIG. 10 may be performed by the components of a headset (e.g., the headset 100). Other entities (e.g., components of the frame 205 and the healthcare platform 800) may perform some or all of the steps of the process 1000 in other embodiments. Likewise, embodiments may include different and/or additional steps, or perform the steps in different orders.

The headset tracks 1005 an eyelid of an eye of a user by a sensor assembly coupled to a frame of a headset. The sensor assembly may comprise at least one light emission element and at least one photodiode. The headset may further comprise a reflector element mounted on the frame that is configured to reflect light emitted from the sensor assembly towards an eye box of the eye, and redirect light reflected from the eyelid towards the sensor assembly. In one embodiment, the sensor assembly is clipped onto a temple of the frame. In another embodiment, the sensor assembly is adhered to the temple of the frame. In yet another embodiment, the sensor assembly is embedded into the temple of the frame, e.g., by using an injection molding. At least a portion of the temple behind a hinge of the frame that includes the sensor assembly may be removable. In yet another embodiment, the sensor assembly is embedded into a front side of the frame.

The headset captures 1010 eyelid tracking information at the sensor assembly. The at least one photodiode of the sensor assembly may capture light reflected from the eyelid and/or one or more other surfaces of the eye. The eyelid tracking information may comprise information about intensities of signals related to the reflected light over a time, and an intensity of the captured light signal may be related to a position of the eyelid. In one embodiment, the headset processes (e.g., via a controller coupled to the least one photodiode) the captured eyelid tracking information to determine positions of the eyelid over time based on intensities of the captured light signal. In another embodiment, the captured eyelid tracking information is processed by a secondary device coupled to the headset.

The headset communicates 1015 (e.g., via a transceiver of the headset) the eyelid tracking information from the headset to the secondary device coupled to the headset for processing the eyelid tracking information and determination of sleep information for the user based in part on the processed eyelid tracking information. The processed eyelid tracking information may be combined at the secondary device with information from a sleep tracker of the user for determination of the sleep information for the user. In one embodiment, the determined sleep information may comprise information about a daily sleep need for the user. In another embodiment, the determined sleep information may comprise information about a sleep deprivation for the user and a reaction time of the user. In yet another embodiment, the determined sleep information may comprise at least one of: information about a sleep deprivation for the user, information about a sleep excess for the user, information about a sleep sensitivity for the user, and information about a psychomotor performance (e.g., psychomotor vigilance) for the user.

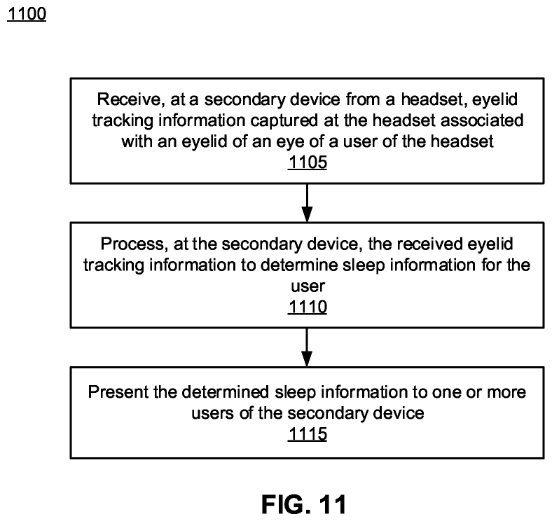

FIG. 11 is a flow chart illustrating a process 1100 performed at a secondary device for determining sleep information for a user of a headset coupled to the secondary device based on eyelid tracking information captured at the headset, in accordance with one or more embodiments. The process 1100 of FIG. 11 may be performed by the components of a secondary device (e.g., the secondary device 810 or the secondary device 910). Other entities may perform some or all of the steps of the process 1100 in other embodiments. Likewise, embodiments may include different and/or additional steps, or perform the steps in different orders.

The secondary device receives 1105 from a headset (e.g., via a transceiver of the secondary device) eyelid tracking information captured at the headset associated with an eyelid of an eye of a user of the headset. The received eyelid tracking information may comprise information about intensities of signals over time related to light reflected from the eyelid and/or at least one other surface of the eye. An intensity of the captured reflected light signal may be related to a position of the eyelid.

The secondary device processes 1110 (e.g., via a controller of the secondary device) the received eyelid tracking information to determine sleep information for the user. The secondary device may process the eyelid tracking information to obtain various eyelid statistics (or blink metrics), e.g., for correlation with the user's psychomotor performance. Examples of the eyelid statistics may include: a blink duration, PERCLOS, an eyelid closing duration, an eyelid closing speed, a duration of eyelid being closed, an eyelid reopening duration, an eyelid reopening speed, a blink frequency, a blink interval, some other eyelid statistics, or some combination thereof. The secondary device may process the received eyelid tracking information, e.g., by evaluating intensities of the reflected light signals captured over time indicating position changes of the eyelid over time. The secondary device may combine the processed eyelid tracking information with information from a sleep tracker worn by the user to determine the sleep information. The sleep information may comprise information about, e.g., a daily sleep need for the user, information about a sleep sensitivity for the user, information about a psychomotor performance for the user, some other sleep information, or some combination thereof.

The secondary device presents 1115 (e.g., via a display of the secondary device) the determined sleep information to one or more users of the device.

### Additional Configuration Information

The foregoing description of the embodiments has been presented for illustration; it is not intended to be exhaustive or to limit the patent rights to the precise forms disclosed. Persons skilled in the relevant art can appreciate that many modifications and variations are possible considering the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A headset comprising:
a sensor assembly coupled to a frame of the headset, the sensor assembly configured to:
track an eyelid of an eye of a user, and
capture eyelid tracking information; and
a transceiver coupled to the sensor assembly, the transceiver configured to:
obtain the eyelid tracking information from the sensor assembly, and
communicate the eyelid tracking information to a secondary device coupled to the headset for processing the eyelid tracking information and determination of sleep information for the user based in part on the processed eyelid tracking information.

2. The headset of claim 1, wherein the determined sleep information comprises information about a daily sleep need for the user.

3. The headset of claim 1, wherein the determined sleep information comprises information about a sleep deprivation for the user and a reaction time of the user.

4. The headset of claim 1, wherein the determined sleep information comprises at least one of: information about a sleep deprivation for the user, information about a sleep excess for the user, information about a sleep sensitivity for the user, and information about a psychomotor performance for the user.

5. The headset of claim 1, wherein the processed eyelid tracking information is combined at the secondary device with information from a sleep tracker of the user for determination of the sleep information for the user.

6. The headset of claim 1, further comprising an ambient light sensor mounted on the frame, the ambient light sensor configured to:
capture information about a spectrum of light incident on the eye,
wherein the captured spectrum information is provided to the secondary device for processing and presentation to the user as part of the sleep information.

7. The headset of claim 1, wherein the sensor assembly is clipped onto a temple of the frame.

8. The headset of claim 1, wherein the sensor assembly is adhered to a temple of the frame.

9. The headset of claim 1, wherein the sensor assembly is embedded into a temple of the frame.

10. The headset of claim 9, wherein the sensor assembly is embedded into the temple using an injection molding.

11. The headset of claim 9, wherein at least a portion of the temple behind a hinge of the frame that includes the sensor assembly is removable.

12. The headset of claim 1, wherein the sensor assembly is embedded into a front side of the frame.

13. The headset of claim 1, further comprising a reflector element mounted on the frame, the reflector element configured to:
reflect light emitted from the sensor assembly towards an eye box of the eye; and
redirect light reflected from the eyelid towards the sensor assembly.

14. The headset of claim 13, further comprising a display element configured to emit image light, and the reflector element is further configured to project the image light to the eye.

15. A method comprising:
tracking an eyelid of an eye of a user by a sensor assembly coupled to a frame of a headset;
capturing eyelid tracking information at the sensor assembly; and
communicating the eyelid tracking information from the headset to a secondary device coupled to the headset for processing the eyelid tracking information and determination of sleep information for the user based in part on the processed eyelid tracking information.

16. The method of claim 15, wherein the determined sleep information comprises at least one of: information about a sleep deprivation for the user, information about a sleep excess for the user, information about a sleep sensitivity for the user, and information about a psychomotor performance for the user.

17. The method of claim 15, further comprising:
capturing, by an ambient light sensor mounted on the frame, information about a spectrum of light incident on the eye; and
providing the captured spectrum information to the secondary device for processing and presentation to the user as part of the sleep information.

18. A method comprising:
receiving, at a device from a headset, eyelid tracking information captured at the headset associated with an eyelid of an eye of a user of the headset;
processing the received eyelid tracking information to determine sleep information for the user; and
presenting the determined sleep information to one or more users of the device.

19. The method of claim 18, wherein determining the sleep information comprises determining, based in part on the processed eyelid tracking information, at least one of: information about a sleep deprivation for the user, information about a sleep excess for the user, information about a sleep sensitivity for the user, and information about a psychomotor performance for the user.

20. The method of claim 18, further comprising:
combining the processed eyelid tracking information with information from a sleep tracker of the user to determine information about a daily sleep need for the user and information about a sleep sensitivity for the user.
